(19) United States
(12) Patent Application Publication
(10) Pub. No.: US 2024/0415767 A1
(43) Pub. Date: Dec. 19, 2024

(54) Tattoo Aftercare Sealant Gel

(71) Applicant: Brent Paul Spicer, Cleveland, OH (US)

(72) Inventor: Brent Paul Spicer, Cleveland, OH (US)

(21) Appl. No.: 18/742,931

(22) Filed: Jun. 13, 2024

Related U.S. Application Data
(60) Provisional application No. 63/507,761, filed on Jun. 13, 2023.

(51) Int. Cl. A61K 9/06; A61K 31/047; A61K 31/12; A61K 31/355; A61K 31/4166; A61K 35/644; A61K 36/185; A61K 36/355; A61K 36/489; A61K 36/53; A61K 36/534; A61K 36/537; A61K 36/82; A61K 36/886; A61K 36/889; A61K 47/32; A61P 17/02

(52) U.S. Cl. CPC A61K 9/06; A61K 31/047; A61K 31/12; A61K 31/355; A61K 31/4166; A61K 35/644; A61K 36/185; A61K 36/355; A61K 36/489; A61K 36/53; A61K 36/534; A61K 36/537; A61K 36/5777; A61K 36/82; A61K 36/886; A61K 36/889; A61K 47/32; A61P 17/02

(57) ABSTRACT

The present invention relates to compositions or formulations or systems for aftercare of a recently-created tattoo on a skin, that includes a tattoo aftercare sealant gel. The tattoo aftercare sealant gel comprises gel-forming-and-bonding components, and natural ingredients for healing to alleviate the risk of wounding or scarring or infection or drying of the skin or irritating the skin, and to keep the tattoo from fading or drying out. Further, the present invention also discloses methods of production of the compositions or formulations, or systems for aftercare of a recently-created tattoo on a skin as disclosed herein, and it creates a tattoo aftercare sealant gel of the present invention. Also, the present invention provides methods of treatment and/or prevention of wounds, injury, infection and/or disease of tattooed skin, for aftercare of a recently-created tattoo on a skin, involving the use of the tattoo aftercare sealant gel of the present invention.

### FIELD OF THE INVENTION

The present invention relates generally to compositions or formulations, systems; methods of production of said compositions or formulations, or systems; and methods of treatment and/or prevention of wounds, injury, infection and/or disease of tattooed skin post-tattooing comprising said compositions or formulations, or systems, for aftercare of a recently-created tattoo on a skin. More specifically, the present invention relates to compositions or formulations, or systems; methods of production of said compositions or formulations, or systems; and methods of treatment and/or prevention of wounds, injury, infection and/or disease of tattooed skin comprising said compositions or formulations, or systems, for aftercare of a recently-created tattoo on a skin, comprising a tattoo aftercare sealant.

### BACKGROUND OF THE INVENTION

Tattoos are a popular form of body art that has existed for centuries. Modern tattoos are applied using needles and ink that penetrate the skin of a subject. The tattoo process creates countless micro-punctures in the dermis into which ink is deposited for the purpose of creating a permanent tattoo. The dermis, located below the epidermis, is the sensitive connective tissue layer of the skin which contains nerve endings, sweat, sebaceous glands, blood and lymph vessels. The tattoo needle deposits particles of pigment into the dermis to create a permanent tattoo.

The ink deposited in the dermis may escape through the micro-punctures on the dermis. This loss of ink may potentially limit or decrease the technical quality and visual appeal of the tattoo. In addition, the ink seeping from the fresh tattoo may stain clothes or any other surface the ink contacts. Several hours after the tattoo has been created, continued bleeding and wound exudation may contribute to the loss of ink from the tattoo. Exudate fluid is generally composed of cells, platelets, proteins, serum, white blood cells, red blood cells, and similar body matter or fluids. The flow of exudate away from the wound may also reduce infection. However, as the exudate dries, a crust is formed that not only depletes the moist wound healing environment but also tears away the adherent exudate from the wound. The removal of the exudative crust commonly results in secondary injury to the wound as well as ink loss in the case of tattoo wounds. The best wound covers keep the wound moist while also absorbing exudates to expedite healing of the wound. A moist wound healing environment accelerates the recovery time and also reduces the amount of scarring on the wounded tissue.

The tattooing industry, and the techniques used therein, has been around for hundreds of years in one form or another. The various techniques used for tattooing have not changed significantly over time. The aftercare that takes care of the tattooed skin against fading, smearing, quality maintenance, injury, infection, disease etc., after creation of a fresh tattoo, protects against the aforementioned exudate formation, and promotes healing as used in the tattooing industry has also not changed significantly, and it ranges from doing nothing, to applying homemade lotions, or balms, to the tattoo. Each tattoo artist has his or her own technique for aftercare.

Furthermore, for a tattoo wound, the formation of a scab or exudative crust is highly undesirable and detrimental to the freshly applied tattoo art. Scabs also form a barrier to the generation of new tissue. Skin regenerates much faster and the risk of infection from airborne pathogens is lower with a moist wound. The premature removal of a scab or exudative crust can cause injury to the healing wound as well as potential loss of tattoo pigment, which is highly undesirable after the pain and expense of obtaining the tattoo.

Considering the above problems post-tattooing, tattoo parlors have developed and routinely use a variety of methods to address wounds created by a fresh tattoo. Existing wound care management of tattoo-created wounds usually consists of the application of petroleum-based products to create an occlusive layer over the wound as well as a secondary dressing, most often not of medical grade quality. Typical secondary dressings used for tattoos are plastic wrap for food or gauze. These commonly available household materials do not absorb wound exudate and may fail to control bleeding. These traditional wound dressing materials also fail to adequately prevent ink loss. The plastic wrap creates a seal around the skin, preventing oxygen from permeating the dressing and reaching the wound and thereby trapping blood and wound exudates against the surface of the wounded skin. Additionally, the body fluids and lost ink become trapped against the skin and are heated by body heat. This moist and warm environment is an ideal breeding ground for bacterial growth and infection.

Another method of treating the wound is to use acrylate polymers. Although acrylate polymers, similar in function to superglue or Dermabond, do not require a secondary dressing, they are difficult to remove and may remain affixed to the wound for days or weeks. Unfortunately, acrylate polymers are also very painful to remove from the skin and wounded area making them undesirable for a tattoo wound.

Recently, hydrogels have been used as a dressing for wound care. Hydrogels are comprised of materials that create intermolecular entanglements using water for dispersal. Hydrogels are highly absorbent natural or synthetic polymers, with high water content, that possess a degree of flexibility similar to natural tissue. Due to their high-water volume, hydrogels can keep a wound moist yet still absorb exudate. While non-crosslinked hydrogels can be spread over a surface with irregular boundaries, the outer surface remains sticky, requiring a secondary dressing to prevent random debris from clinging to the wound covering. Also, traditional hydrogels are not optimal for a patient who is not immobilized. A mobile patient would quickly displace a traditional hydrogel dressing that was not secured by a secondary dressing since the sticky surface may adhere to outside objects and become displaced. Particles may become attached to the sticky surface or the hydrogel may inadvertently be dislodged from the wounded area if the patient rubs the hydrogel against a surface. Thus, in such systems and methods of aftercare, secondary dressings become necessary to prevent these issues and maintain the position of the traditional hydrogel dressing. Alternatively, hydrogels can be cross-linked by radiation, free radicals, or other means to result in a rubbery material that can be cut into various forms but a secondary dressing will still be required to secure the material to the wound and surrounding skin. These rubberized hydrogels are available in strips that may need trimming to accommodate an irregularly shaped wound. Also, in the case of an especially large wound, an impractically high number of strips may be required.

Thus, there exists a problem and need in the field for improved compositions or formulations, or systems for aftercare of a recently-created tattoo on a skin composition and for improved methods of treatment and/or prevention of wounds, injury, infection and/or disease of tattooed skin.

The present invention addresses the above-discussed problem and need by providing compositions or formulations, or systems, for aftercare of a recently-created tattoo on a skin, methods of their production, and methods of treatment and/or prevention of wounds, injury, infection and/or disease of tattooed skin with the use of the presently disclosed compositions or formulations, or systems, for aftercare of a recently-created tattoo on a skin, said compositions or formulations, or systems comprising a tattoo aftercare sealant.

### SUMMARY OF THE INVENTION

The following listing of embodiments is a nonlimiting statement of various aspects of the invention. Other aspects and variations will be evident in light of the entire disclosure.

An aspect of the present invention provides a composition or formulation for aftercare of a recently-created tattoo on a skin, wherein the composition or formulation comprises: a tattoo aftercare sealant gel, said tattoo aftercare sealant gel comprising: gel-forming-and-bonding components; and natural ingredients for healing, wherein the gel-forming-and-bonding components are selected from a group of gel-forming-and-bonding components consisting of: water-soluble or hydrophilic polymers, stabilizers, thickeners, pressure-sensitive adhesives, superabsorbent polymers, softeners, moisturizers, conditioners, emollients, humectants, texture enhancers, solvents, penetration boosters, and any combination thereof, and wherein the natural ingredients for healing are plant-derived natural ingredients.

Another aspect of the present invention provides systems for aftercare of a recently-created tattoo on a skin, wherein the systems comprise the composition or formulation as disclosed herein, comprising a tattoo aftercare sealant gel, wherein said tattoo aftercare sealant gel comprises: gel-forming-and-bonding components; and natural ingredients for healing.

Another aspect of the present invention provides a method of production of the composition or formulation or systems as disclosed herein, the method comprising the steps of: combining gel-forming-and-bonding components to obtain a combination A; combining natural ingredients for healing to obtain a combination B; and combining the combination A and the combination B to obtain a tattoo aftercare sealant gel, wherein the tattoo aftercare sealant gel forms the composition or formulation for aftercare of a recently-created tattoo on a skin.

Another aspect of the present invention provides a method treatment and/or prevention of wounds, injury, infection and/or disease of tattooed skin, the method comprising the steps of: sterilizing a skin area of a subject for tattoo creation by a tattoo artist; creating a tattoo on said skin by said artist; administering to the subject a therapeutically effective amount of the composition or formulation for aftercare of a recently-created tattoo on a skin as disclosed in the present disclosure, wherein the composition or formulation comprises a tattoo aftercare sealant gel, said tattoo aftercare sealant gel comprising: gel-forming-and-bonding components; and natural ingredients for healing, wherein the gel-forming-and-bonding components are selected from a group of gel-forming-and-bonding components consisting of: water-soluble or hydrophilic polymers, stabilizers, thickeners, pressure-sensitive adhesives, superabsorbent polymers, softeners, moisturizers, conditioners, emollients, humectants, texture enhancers, solvents, penetration boosters, and any combination thereof, wherein the natural ingredients for healing are plant-derived natural ingredients, wherein the administering step involves applying the therapeutically effective amount in form of a liquid gel of the composition or formulation comprising said tattoo aftercare sealant gel as a thin layer over the skin where the tattoo was created, covering all such skin in one go and forming a protective layer over such skin, wherein the protective layer can be easily removed, peeled away, or washed off with water after a period of time following the applying of said compositions or formulations or systems comprising said tattoo aftercare sealant gel, and wherein said period of time can range from hours to days post the administering step.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The accompanying drawings are included to provide a further understanding of the invention, and are incorporated in and constitute a part of the present invention and, together with the description, serve to explain the principle of the invention.

In the drawings,

FIG. 1 depicts a schematic of a method of making or producing a combination or formulation for aftercare of a recently-created tattoo on a skin in an embodiment of the present invention, comprising a tattoo aftercare sealant gel.

FIG. 2 illustrates an embodiment of present invention and illustrates an exemplary schematic spanning over the process of (a) creating a tattoo by an artist to (b) applying combinations or compositions or formulations or systems comprising said tattoo aftercare sealant gel as disclosed in the present disclosure of the present invention and (c) post-application of said tattoo aftercare sealant gel.

FIG. 3 illustrates an embodiment of present invention and illustrates an exemplary schematic for a tattoo aftercare sealant gel as disclosed in the present disclosure of the present invention in a liquid form before applying it over a freshly applied tattoo on the skin of a subject.

FIG. 4 illustrates an embodiment of present invention and illustrates an exemplary schematic for a tattoo aftercare sealant gel as disclosed in the present disclosure of the present invention applied wet over a freshly applied tattoo on the skin of a subject covering the whole expanse of the tattooed area of the skin of the subject with a thin, transparent, shiny layer.

FIG. 5 illustrates an embodiment of present invention and illustrates an exemplary schematic for a tattoo aftercare sealant gel as disclosed in the present disclosure of the present invention applied wet and drying forming a protective seal covering the whole expanse of the tattooed area of the skin of the subject.

FIG. 6 illustrates an embodiment of present invention and illustrates an exemplary schematic for a tattoo aftercare sealant gel as disclosed in the present disclosure of the present invention being removed as a hardened gel after several hours following initial applying of the tattoo aftercare sealant gel as combinations or compositions or formulations or systems for aftercare of a recently-created tattoo immediately following the creation of the tattoo on a skin by insertion of ink creating said tattoo.

### DETAILED DESCRIPTION OF THE INVENTION

Detailed embodiments of the present invention are disclosed herein. However, it is to be understood that the disclosed embodiments are merely exemplary of the present invention, which may be embodied in various systems. Therefore, specific details disclosed herein are not to be interpreted as limiting, but rather as basis for teaching one skilled in the art to variously practice the present invention.

All illustrations of the drawings are for the purpose of describing selected versions of the present invention and are not intended to limit the scope of the present invention.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

Although any methods and materials similar or equivalent to those described herein can be used in the practice of the present invention, the exemplary methods, devices, and materials are described herein. For the purposes of the present disclosure, the following terms are defined below. Additional definitions are set forth throughout this disclosure.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a microbe, a microbial formulation, a pharmaceutical composition, and/or a method that “comprises” a list of elements (e.g., components, features, or steps) is not necessarily limited to only those elements (or components or steps), but may include other elements (or components or steps) not expressly listed or inherent to the microbe, microbial formulation, pharmaceutical composition and/or method. Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “and/or” when used in a list of two or more items, means that any one of the listed items can be employed by itself or in combination with any one or more of the listed items. For example, the expression “A and/or B” is intended to mean either or both of A and B, i.e., A alone, B alone or A and B in combination. The expression “A, B and/or C” is intended to mean A alone, B alone, C alone, A and B in combination, A and C in combination, B and C in combination or A, B, and C in combination.

As discussed above, there remains a problem and need in the art for improved tattoo aftercare and to provide a solution to address that, the present invention provides compositions or formulations, or systems; methods of production of said compositions or formulations, or systems; and methods of treatment and/or prevention of wounds, injury, infection and/or disease of tattooed skin, comprising administration of said compositions or formulations, or systems, for aftercare of a recently-created tattoo on a skin, wherein said compositions or formulations, or systems comprise a tattoo aftercare sealant gel to alleviate the risk of wounding or scarring or infection or drying of the skin or irritating the skin, and to keep the tattoo from fading or drying out.

In an embodiment of the present invention, it provides a composition or formulation for aftercare of a recently-created tattoo on a skin, wherein the composition or formulation comprises: a tattoo aftercare sealant gel, said tattoo aftercare sealant gel comprising: gel-forming-and-bonding components; and natural ingredients for healing, wherein the gel-forming-and-bonding components are selected from a group of gel-forming-and-bonding components consisting of: water-soluble or hydrophilic polymers, stabilizers, thickeners, pressure-sensitive adhesives, superabsorbent polymers, softeners, moisturizers, conditioners, emollients, humectants, texture enhancers, solvents, penetration boosters, and any combination thereof, and wherein the natural ingredients for healing are plant-derived natural ingredients.

In another embodiment of the present invention providing the composition or formulation for aftercare of a recently-created tattoo on a skin as disclosed herein, wherein the plant-derived natural ingredients are selected from a group consisting of: antioxidant agents, skin-soothing agents, anti-inflammatory agents, anti-cancer agents, skin-conditioning agents, skin-healing agents, pH adjuster agents, emulsifier agents, surfactant agents, fragrance agents, skin-soothing agents, moisturizing agents, keratolytic agents, antimicrobial agents, antifungal agents, antiviral agents, skin-cleansing agents, skin-regenerative agents, wound healing agents, anti-redness agents, anti-swelling agents, skin-exfoliating agents, and any combination thereof.

In an embodiment of the present invention provides a composition or formulation for use in aftercare of a recently-created tattoo on a skin, wherein the composition or formulation comprises: a tattoo aftercare sealant gel, said tattoo aftercare sealant gel comprising: gel-forming-and-bonding components; and natural ingredients for healing, wherein the gel-forming-and-bonding components are selected from a group of gel-forming-and-bonding components consisting of: water-soluble or hydrophilic polymers, stabilizers, thickeners, pressure-sensitive adhesives, superabsorbent polymers, softeners, moisturizers, conditioners, emollients, humectants, texture enhancers, solvents, penetration boosters, and any combination thereof, and wherein the natural ingredients for healing are plant-derived natural ingredients.

The composition or formulation for use in aftercare of a recently-created tattoo on a skin as disclosed herein, wherein the plant-derived natural ingredients are selected from a group consisting of: antioxidant agents, skin-soothing agents, anti-inflammatory agents, anti-cancer agents, skin-conditioning agents, skin-healing agents, pH adjuster agents, emulsifier agents, surfactant agents, fragrance agents, skin-soothing agents, moisturizing agents, keratolytic agents, antimicrobial agents, antifungal agents, antiviral agents, skin-cleansing agents, skin-regenerative agents, wound healing agents, anti-redness agents, anti-swelling agents, skin-exfoliating agents, and any combination thereof.

In an embodiment of the present invention, it provides systems for aftercare of a recently-created tattoo on a skin, wherein the systems comprise the composition or formulation comprising a tattoo aftercare sealant gel, said tattoo aftercare sealant gel comprising: gel-forming-and-bonding components; and natural ingredients for healing.

In another embodiment of the present invention providing the systems for aftercare of a recently-created tattoo on a skin as disclosed herein, wherein the gel-forming-and-bonding components are selected from a group of gel-forming-and-bonding components consisting of: water-soluble or hydrophilic polymers, stabilizers, thickeners, pressure-sensitive adhesives, superabsorbent polymers, softeners, moisturizers, conditioners, emollients, humectants, texture enhancers, solvents, penetration boosters, and any combination thereof.

In another embodiment of the present invention providing the systems for aftercare of a recently-created tattoo on a skin as disclosed herein, wherein the natural ingredients for healing are plant-derived natural ingredients, and wherein the plant-derived natural ingredients are selected from a group consisting of: antioxidant agents, skin-soothing agents, anti-inflammatory agents, anti-cancer agents, skin-conditioning agents, skin-healing agents, pH adjuster agents, emulsifier agents, surfactant agents, fragrance agents, skin-soothing agents, moisturizing agents, keratolytic agents, antimicrobial agents, antifungal agents, antiviral agents, skin-cleansing agents, skin-regenerative agents, wound healing agents, anti-redness agents, anti-swelling agents, skin-exfoliating agents, and any combination thereof.

In an embodiment of the present invention, it provides systems for use in aftercare of a recently-created tattoo on a skin, wherein the systems comprise the composition or formulation comprising a tattoo aftercare sealant gel, said tattoo aftercare sealant gel comprising: gel-forming-and-bonding components; and natural ingredients for healing.

In another embodiment of the present invention providing the systems for use in aftercare of a recently-created tattoo on a skin as provided herein, wherein the gel-forming-and-bonding components are selected from a group of gel-forming-and-bonding components consisting of: water-soluble or hydrophilic polymers, stabilizers, thickeners, pressure-sensitive adhesives, superabsorbent polymers, softeners, moisturizers, conditioners, emollients, humectants, texture enhancers, solvents, penetration boosters, and any combination thereof.

In another embodiment of the present invention providing the systems for use in aftercare of a recently-created tattoo on a skin as provided herein, wherein the natural ingredients for healing are plant-derived natural ingredients, and wherein the plant-derived natural ingredients are selected from a group consisting of: antioxidant agents, skin-soothing agents, anti-inflammatory agents, anti-cancer agents, skin-conditioning agents, skin-healing agents, pH adjuster agents, emulsifier agents, surfactant agents, fragrance agents, skin-soothing agents, moisturizing agents, keratolytic agents, antimicrobial agents, antifungal agents, antiviral agents, skin-cleansing agents, skin-regenerative agents, wound healing agents, anti-redness agents, anti-swelling agents, skin-exfoliating agents, and any combination thereof.

In an embodiment of the present invention, it provides a method of production of the composition or formulation for aftercare of a recently-created tattoo on a skin as disclosed herein the present disclosure, the method comprising the steps of: combining gel-forming-and-bonding components to obtain a combination A; combining natural ingredients for healing to obtain a combination B; and combining the combination A and the combination B to obtain a tattoo aftercare sealant gel, wherein said tattoo aftercare sealant gel forms the composition or formulation for aftercare of a recently-created tattoo on a skin.

In another embodiment of the present invention providing the method of production of the composition or formulation for aftercare of a recently-created tattoo on a skin as disclosed herein, wherein the gel-forming-and-bonding components are selected from a group of gel-forming-and-bonding components consisting of: water-soluble or hydrophilic polymers, stabilizers, thickeners, pressure-sensitive adhesives, superabsorbent polymers, softeners, moisturizers, conditioners, emollients, humectants, texture enhancers, solvents, penetration boosters, and any combination thereof.

In another embodiment of the present invention providing the method of production of the composition or formulation for aftercare of a recently-created tattoo on a skin as disclosed herein, wherein the natural ingredients for healing are plant-derived natural ingredients, and wherein the plant-derived natural ingredients are selected from a group consisting of: antioxidant agents, skin-soothing agents, anti-inflammatory agents, anti-cancer agents, skin-conditioning agents, skin-healing agents, pH adjuster agents, emulsifier agents, surfactant agents, fragrance agents, skin-soothing agents, moisturizing agents, keratolytic agents, antimicrobial agents, antifungal agents, antiviral agents, skin-cleansing agents, skin-regenerative agents, wound healing agents, anti-redness agents, anti-swelling agents, skin-exfoliating agents, and any combination thereof.

In an embodiment of the present invention, it provides a method of treatment and/or prevention of wounds, injury, infection and/or disease of tattooed skin, the method comprising the steps of: sterilizing a skin area of a subject for tattoo creation by a tattoo artist; creating a tattoo on said skin by said artist; administering to the subject a therapeutically effective amount of the composition or formulation for aftercare of a recently-created tattoo on a skin as disclosed in the present disclosure, wherein the composition or formulation comprise a tattoo aftercare sealant gel, said tattoo aftercare sealant gel comprising: gel-forming-and-bonding components; and natural ingredients for healing, wherein the gel-forming-and-bonding components are selected from a group of gel-forming-and-bonding components consisting of: water-soluble or hydrophilic polymers, stabilizers, thickeners, pressure-sensitive adhesives, superabsorbent polymers, softeners, moisturizers, conditioners, emollients, humectants, texture enhancers, solvents, penetration boosters, and any combination thereof, and wherein the natural ingredients for healing are plant-derived natural ingredients, wherein the administering step involves applying the therapeutically effective amount in form of a liquid gel of the compositions or formulations or systems comprising said tattoo aftercare sealant gel as a thin layer over the skin where the tattoo was created, covering all such skin in one go and forming a protective layer over such skin, wherein the protective layer can be easily removed, peeled away, or washed off with water after a period of time following the applying of said compositions or formulations or systems comprising said tattoo aftercare sealant gel, and wherein said period of time can range from hours to days post the administering step.

In another embodiment of the present invention providing the method treatment and/or prevention of wounds, injury, infection and/or disease of tattooed skin as disclosed herein, wherein the gel-forming-and-bonding components are selected from a group of gel-forming-and-bonding components consisting of: water-soluble or hydrophilic polymers, stabilizers, thickeners, pressure-sensitive adhesives, superabsorbent polymers, softeners, moisturizers, conditioners, emollients, humectants, texture enhancers, solvents, penetration boosters, and any combination thereof.

In another embodiment of the present invention providing the method treatment and/or prevention of wounds, injury, infection and/or disease of tattooed skin as disclosed herein, wherein the natural ingredients for healing are plant-derived natural ingredients, and wherein the plant-derived natural ingredients are selected from a group consisting of: antioxidant agents, skin-soothing agents, anti-inflammatory agents, anti-cancer agents, skin-conditioning agents, skin-healing agents, pH adjuster agents, emulsifier agents, surfactant agents, fragrance agents, skin-soothing agents, moisturizing agents, keratolytic agents, antimicrobial agents, antifungal agents, antiviral agents, skin-cleansing agents, skin-regenerative agents, wound healing agents, anti-redness agents, anti-swelling agents, skin-exfoliating agents, and any combination thereof.

The terms “subject,” “patient” and “individual” as used herein are used interchangeably herein to refer to a vertebrate, including mammals and humans. A “subject,” “patient” or “individual” as used herein, includes any animal that exhibits pain that can be treated with the compositions or formulations or systems, and methods contemplated herein, and that includes laboratory animals, farm animals, and domestic animals or pets, non-human primates and human are included.

As used herein, the term “amount” refers to “an amount effective” or “an effective amount” of a cell to achieve a beneficial or desired prophylactic or therapeutic result, including clinical results.

As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g., a symptom associated with the disease or condition being treated. As such, “treatment” also includes situations where the disease, disorder, or pathological condition, or at least symptoms associated therewith, are completely inhibited (e.g., prevented from happening) or stopped (e.g., terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent. In some embodiments, the engineered cell or pharmaceutical composition comprising said engineered cell of the disclosure is administered in a prophylactically effective amount.

In the tattoo aftercare sealant gel of the present disclosure as disclosed herein, the gel-forming-and-bonding components are the primary bonding agents that allow the gel to dry and form a protective layer over the tattoo. Further, in the tattoo aftercare sealant gel of the present disclosure as disclosed herein, the natural ingredients for healing that are plant-based products have been selected for their proven healing and soothing properties.

The present invention in other words provides a liquid gel that is applied to the skin after a fresh tattoo. The gel dries forming a protective layer over the fresh tattoo preventing infection and helping the healing process with natural ingredients.

In the process of applying the compositions or formulations or systems for aftercare of a tattoo and methods of treatment and/or prevention of wounds, injury, infection and/or disease of tattooed skin as disclosed herein, the protective layer or skin can be peeled away or washed off with water hours later.

In the presently disclosed compositions or formulations or systems for aftercare of a tattoo and methods of treatment and/or prevention of wounds, injury, infection and/or disease of tattooed skin as disclosed herein, a tattoo aftercare sealant gel, said tattoo aftercare sealant gel comprising: gel-forming-and-bonding components; and natural ingredients for healing, also as disclosed herein, the tattoo aftercare sealant gel forms a layer that prevents dirt and bacteria from entering the tattoo and reduces plastic and waste. It easy and convenient and a substitute to plastic and traditional bandages.

The products and methods as disclosed herein for the present invention are advantageous as compared to other aftercare products available in the art either commercially or otherwise, which are often creams and balms and none of these products contain the same properties or functionality as that of the presently disclosed products and/or methods as disclosed herein.

Moreover, there are many aftercare products and method conventionally available in the art but none of them with a sealant gel. The presently disclosed products and methods comprising the presently disclosed tattoo aftercare sealant gel, said tattoo aftercare sealant gel comprising: gel-forming-and-bonding components; and natural ingredients for healing, have the ability to go on wet and cover the complete tattooed surface, then dry, sealing the tattoo from germs, dirt, bacteria, and other pathogens and irritants, making it the only product of its kind in the art. There is also a substantial reduction in waste from the use of the products and in the methods of the present disclosure than with plastic and other traditional coverings which are also used to treat tattoos, making it environment and pocket friendly. Even more advantageously, the presently disclosed products and methods of the present invention are convenient to use, soothe the skin, and produce zero waste from each application, and have an ease of use and of application making it malleable for use by anyone dealing with a tattoo on a skin.

The invention will be further explained by the following Examples, which are intended to purely exemplary of the invention, and should not be considered as limiting the invention in any way.

### EXAMPLES

### Example 1

### Method of Making the Compositions or Formulations or Systems of a Liquid Gel Sealant for Tattoo Aftercare

This example provides exemplary method of making the composition or formulation for systems and for use in form of a liquid gel sealant of the present invention for tattoo aftercare of an embodiment of the present invention. The method comprises the use of the following components in the below listed amounts as shown in the Table 1 herein below.

TABLE 1
Recipe for a combination or formulation or system
for aftercare of a recently-created tattoo on a skin
as per the embodiment of the present invention:

| | Percent amount in the |
|---|---|
| | combination or formulation |
| Ingredient or component | or system out of 100 |
| Polyvinyl alcohol | 15 |
| Acrylates copolymer | 6 |
| Butylene glycol | 5 |
| Rosmarinus officinalis (rosemary) | 5 |
| extract | |
| Portulaca oleracea extract | 2 |
| PEG-40 hydrogenated castor oil | 1 |
| Lonicera japonica extract | 1 |
| Aloe barbadensis leaf extract | 1 |
| 1,2-hexanediol | 0.6 |
| Hydroxyacetophenone | 0.6 |
| Mentha arvensis extract | 0.5 |
| Sophora angustifolia root extract | 0.5 |
| Triethanolamine | 0.4 |
| Allantoin | 0.2 |
| Lavandula angustifolia (lavender) oil | 0.1 |
| Tocopherol | 0.1 |
| Theobroma cacao (cocoa) seed butter | 0.1 |
| Shea butter | 1 |
| Aloe butter coconut oil | 1 |
| Green tea | 1 |
| Beeswax | 1 |
| Alcohol | 12 |
| Water | 44.9 |
| Total | 100 |

In this example, the following components and ingredients have been used because of their following characteristics and properties:

Polyvinyl alcohol is a water-soluble synthetic polymer. It is used in papermaking, textile warp sizing, as a thickener and emulsion stabilizer in polyvinyl acetate adhesive formulations, it is also used in contact lenses and face masks.

Acrylate polymer adds strength to the product to reduce tears or cuts on the gel while it is dry, commonly used in beauty products such as nail polish as an adhesive.

Butylene glycol is a skin moisturizer and conditioner.

Portulaca oleracea is an antioxidant and offers skin-soothing benefits.

Peg-40 hydrogenated castor oil is used in cosmetics and beauty products as an emulsifier, surfactant, and fragrance ingredient.

Lonicera japonica extract (honeysuckle extract) is an antioxidant.

Aloe barbadensis leaf extract (aloe vera extract) is used to sooth irritated or mildly damaged skin.

1,2-hexanediol is a moisture-binding chemical and it also has some antioxidant, anti-inflammatory, and anti-cancer benefits.

Hydroxyacetophenone is a synthetic antioxidant and skin-conditioning ingredient.

Mentha arvensis is commonly used as a contraceptive, carminative, antispasmodic, anti-peptic ulcer healing agent.

Sophora angustifolia root extract is an herbal medicine used to treat various skin diseases, fever, and inflammation.

Triethanolamine is used as a pH adjuster and emulsifier in various cosmetic products such as shaving gels and creams, sunscreens, lotions, serums, and cleansers.

Allantoin has been used in skin care products for centuries due to its moisturizing and keratolytic properties. It helps to soften and protect while actively soothing skin.

Rosemary Extract (Rosmarinus officinalis) has anti-inflammatory properties that help to reduce swelling and puffiness of the skin. It also helps to heal burns and soothe the skin. This herb's medicinal qualities are known to make it a powerful cure for chronic skin conditions including dermatitis, eczema and psoriasis.

Lavander Oil (Lavandula angustifolia) is both antimicrobial and antifungal and has anti-inflammatory properties, is known for evening out skin tone and adding a natural glow to skin, and helps reduce redness, blotchy patches, and acne scarring.

Tocopherhol or alpha-tocopherol is the most biologically active form of vitamin E. It helps retain moisture to keep your skin looking radiant and healthy, it can make an excellent moisturizer for dry, patchy areas of skin, and it prevents erythema, edema, skin swelling, and skin thickening.

Theobroma Cacao (Cocoa) and its seed butter effectively enhances skin health by its active free radical scavenging activities and protect the skin from premature aging, also has antioxidative properties, it is effective at preserving skin's moisture balance by restoring the lipidic layer of the skin, and it reduces redness and soothes active acne spots.

Shea Butter helps clear your skin of excess oil (sebum) and also has anti-inflammatory, moisturizing, and healing properties.

Aloe butter is an extract of aloe vera (Aloe barbadensis) in a coconut fatty fraction. It is semi solid at room temperature, melting when applied to the skin. Aloe Butter aids in rapid hydration of dry skin caused by eczema, psoriasis, rosacea, sun burn, wind burn, and general chapping.

Coconut Oil has anti-inflammatory, antibacterial, and antiviral properties, and it may help moisturize your skin, relieve inflammation, and accelerate wound healing.

Green tea has anti-inflammatory properties that can help reduce skin irritation, skin redness, and swelling, it not only moisturizes the skin, but it also brightens and repairs it, and it helps to reverse sun damage and fades dark spots, pimple spots, and other skin irritations caused by environmental aggressors.

Beeswax hydrates, conditions, soothes, and calms the skin. It exfoliates, repairs damage, promotes the skin's regeneration, diminishes the appearance of the signs of aging, soothes itchiness and irritation, and creates a hydrating, long-lasting protective barrier against environmental pollutants.

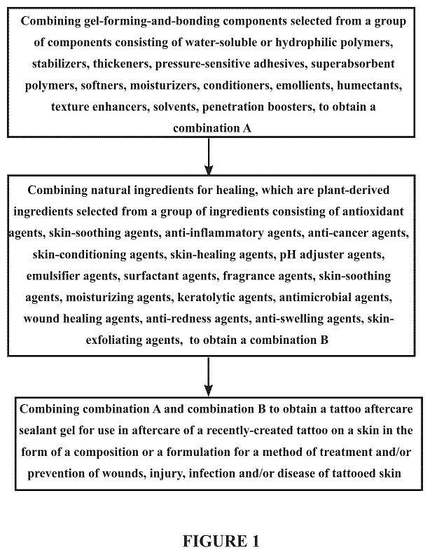

FIG. 1 provides a schematic of an embodiment of a method for making or producing the combination or formulation comprising the tattoo aftercare sealant gel of the present invention.

### Example 2

Method of Treatment and/or Prevention of Wounds, Injury, Infection and/or Disease of Tattooed Skin Using Instant Aftercare as Disclosed Herein

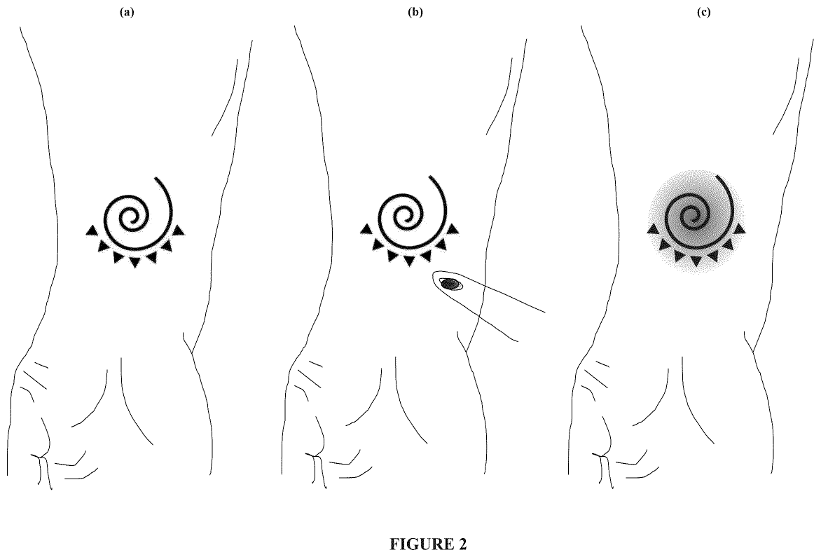

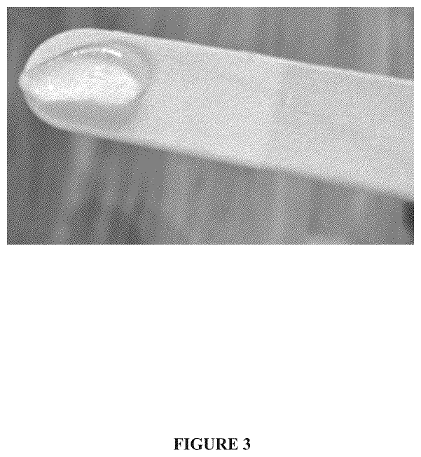

This example provides exemplary method of treatment and/or prevention of wounds, injury, infection and/or disease of tattooed skin as an embodiment of the present disclosure of the present invention. FIG. 2 provides a schematic representation of steps of such method starting at panel (a) showing post-creation of a freshly created tattoo on a skin of a subject (human arm here) followed by panel (b) showing the combination or formulation or system of the present invention as a liquid gel form for application over the freshly created tattoo, said liquid gel form in a real-life example is shown in the illustration of FIG. 3, and panel (c) illustrates the tattooed skin post-application of the said gel for aftercare of the tattoo which is seen as a transparent film formed post-application. The presently disclosed composition or formulation or system improves wound healing for a freshly applied tattoo as well as reduces the formation of exudative crusting and reduces ink loss. The presently disclosed composition or formulation or system also absorbs exudate, adheres to the moist wound surface and can be removed with little or no pain at the wounded skin surface. It can be used on a wound of practically any size or shape due to its accommodating and adaptable nature. The presently disclosed composition or formulation or system and the methods using the same also prevent loss of ink from the tattooed surface and allows the tattoo art to be immediately visible to the person and whoever wants to see the new tattoo. Thus, the present invention provides a unique solution to the above-discussed need and problem in the art.

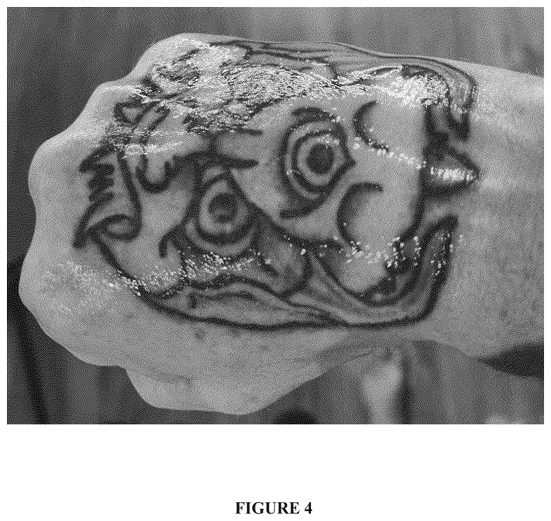

FIG. 4 provides an illustration of a real-life experimental result of application of the compositions or formulations, or systems for aftercare of a recently-created tattoo on a skin as disclosed in the present disclosure after application on a subject (human) of the liquid gel composition or formulation or system of the present invention showing it as wet immediately post-application of the products of the present invention.

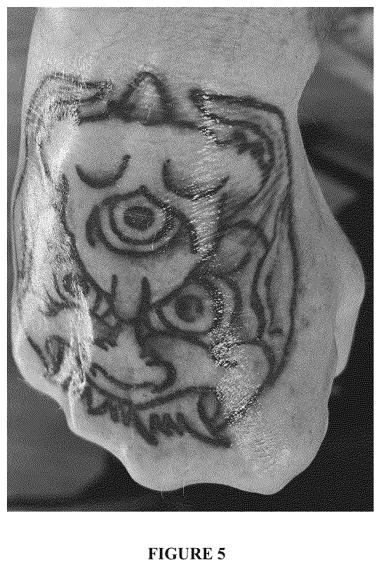

FIG. 5 provides an illustration of a real-life experimental result of application of the compositions or formulations, or systems for aftercare of a recently-created tattoo on a skin as disclosed in the present disclosure after some time having been passed post-application of the products of the present invention where the applied gel dries up from the initial wet as in FIG. 4 to a dried form in FIG. 5, where the said dried layer of the gel sealant for tattoo aftercare of the present invention forms a protective seal over the skin of the tattoo recipient.

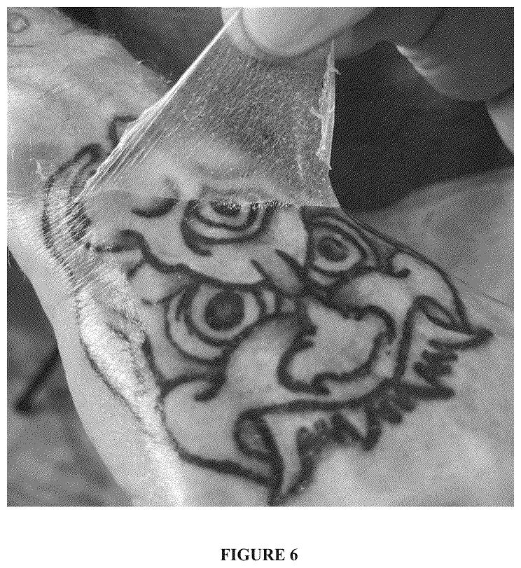

Finally, FIG. 6 provides an illustration of a real-life experimental result of application of the compositions or formulations, or systems for aftercare of a recently-created tattoo on a skin as disclosed in the present disclosure after several hours post-application of the products of the present invention where the dried-up layer as shown in the FIG. 5 is removed as a hardened gel as the last step of the method of treatment and/or prevention of wounds, injury, infection and/or disease of tattooed skin as disclosed herein.

The invention will be further explained by the following Examples, which are intended to purely exemplary of the invention, and should not be considered as limiting the invention in any way.

It will be apparent to those skilled in the art that various modifications and variations can be made in the practice of the present invention without departing from the scope or spirit of the invention. Other embodiments of the invention will be apparent to those skilled in the art from considering of the specification and practice of the invention. It is intended that the specification and examples be considered as exemplary only, with a true scope and spirit of the invention being indicated by the following claims.

1. A composition or formulation for aftercare of a recently-created tattoo on a skin, wherein the composition or formulation comprises:
a tattoo aftercare sealant gel, said tattoo aftercare sealant gel comprising:
gel-forming-and-bonding components; and
natural ingredients for healing,
wherein the gel-forming-and-bonding components are selected from a group of gel-forming-and-bonding components consisting of: water-soluble or hydrophilic polymers, stabilizers, thickeners, pressure-sensitive adhesives, superabsorbent polymers, softeners, moisturizers, conditioners, emollients, humectants, texture enhancers, solvents, penetration boosters, and any combination thereof, and
wherein the natural ingredients for healing are plant-derived natural ingredients.

2. The composition or formulation of claim 1, wherein the plant-derived natural ingredients are selected from a group consisting of: antioxidant agents, skin-soothing agents, anti-inflammatory agents, anti-cancer agents, skin-conditioning agents, skin-healing agents, pH adjuster agents, emulsifier agents, surfactant agents, fragrance agents, skin-soothing agents, moisturizing agents, keratolytic agents, antimicrobial agents, antifungal agents, antiviral agents, skin-cleansing agents, skin-regenerative agents, wound healing agents, anti-redness agents, anti-swelling agents, skin-exfoliating agents, and any combination thereof.

3. The composition or formulation of claim 1, wherein the composition or formulation is comprised in systems for aftercare of a recently-created tattoo on a skin, wherein the systems comprise a tattoo aftercare sealant gel, wherein said tattoo aftercare sealant gel comprises: gel-forming-and-bonding components; and natural ingredients for healing.

4. The composition or formulation of claim 3, wherein the gel-forming-and-bonding components are selected from a group of gel-forming-and-bonding components consisting of: water-soluble or hydrophilic polymers, stabilizers, thickeners, pressure-sensitive adhesives, superabsorbent polymers, softeners, moisturizers, conditioners, emollients, humectants, texture enhancers, solvents, penetration boosters, and any combination thereof.

5. The composition or formulation of claim 3, wherein the natural ingredients for healing are plant-derived natural ingredients, and wherein the plant-derived natural ingredients are selected from a group consisting of: antioxidant agents, skin-soothing agents, anti-inflammatory agents, anti-cancer agents, skin-conditioning agents, skin-healing agents, pH adjuster agents, emulsifier agents, surfactant agents, fragrance agents, skin-soothing agents, moisturizing agents, keratolytic agents, antimicrobial agents, antifungal agents, antiviral agents, skin-cleansing agents, skin-regenerative agents, wound healing agents, anti-redness agents, anti-swelling agents, skin-exfoliating agents, and any combination thereof.

6. A method of production of the composition or formulation for aftercare of a recently-created tattoo on a skin of claim 1, the method comprising the steps of:
combining gel-forming-and-bonding components to obtain a combination A;
combining natural ingredients for healing to obtain a combination B; and
combining the combination A and the combination B to obtain a tattoo aftercare sealant gel, wherein the tattoo aftercare sealant gel forms the composition or formulation for aftercare of a recently-created tattoo on a skin.

7. The method of production of claim 6, wherein the gel-forming-and-bonding components are selected from a group of gel-forming-and-bonding components consisting of: water-soluble or hydrophilic polymers, stabilizers, thickeners, pressure-sensitive adhesives, superabsorbent polymers, softeners, moisturizers, conditioners, emollients, humectants, texture enhancers, solvents, penetration boosters, and any combination thereof.

8. The method of production of claim 6, wherein the natural ingredients for healing are plant-derived natural ingredients, and wherein the plant-derived natural ingredients are selected from a group consisting of: antioxidant agents, skin-soothing agents, anti-inflammatory agents, anti-cancer agents, skin-conditioning agents, skin-healing agents, pH adjuster agents, emulsifier agents, surfactant agents, fragrance agents, skin-soothing agents, moisturizing agents, keratolytic agents, antimicrobial agents, antifungal agents, antiviral agents, skin-cleansing agents, skin-regenerative agents, wound healing agents, anti-redness agents, anti-swelling agents, skin-exfoliating agents, and any combination thereof.

9. A method of treatment and/or prevention of wounds, injury, infection and/or disease of tattooed skin, the method comprising the steps of:
sterilizing a skin area of a subject for tattoo creation by a tattoo artist;
creating a tattoo on said skin by said artist;
administering to the subject a therapeutically effective amount of the composition or formulation for aftercare of a recently-created tattoo on a skin of claim 1,
wherein the composition or formulation comprises a tattoo aftercare sealant gel, said tattoo aftercare sealant gel comprising: gel-forming-and-bonding components; and natural ingredients for healing,
wherein the gel-forming-and-bonding components are selected from a group of gel-forming-and-bonding components consisting of: water-soluble or hydrophilic polymers, stabilizers, thickeners, pressure-sensitive adhesives, superabsorbent polymers, softeners, moisturizers, conditioners, emollients, humectants, texture enhancers, solvents, penetration boosters, and any combination thereof,
wherein the natural ingredients for healing are plant-derived natural ingredients,
wherein the administering step involves applying the therapeutically effective amount in form of a liquid gel of the composition or formulation comprising said tattoo aftercare sealant gel as a thin layer over the skin where the tattoo was created, covering all such skin in one go and forming a protective layer over such skin,
wherein the protective layer can be easily removed, peeled away, or washed off with water after a period of time following the applying of the composition or formulation comprising said tattoo aftercare sealant gel, and
wherein said period of time can range from hours to days post the administering step.

10. The method of treatment and/or prevention of claim 9, wherein the gel-forming-and-bonding components are selected from a group of gel-forming-and-bonding components consisting of: water-soluble or hydrophilic polymers, stabilizers, thickeners, pressure-sensitive adhesives, superabsorbent polymers, softeners, moisturizers, conditioners, emollients, humectants, texture enhancers, solvents, penetration boosters, and any combination thereof.

11. The method of treatment and/or prevention of claim 9, wherein the natural ingredients for healing are plant-derived natural ingredients, and wherein the plant-derived natural ingredients are selected from a group consisting of: antioxidant agents, skin-soothing agents, anti-inflammatory agents, anti-cancer agents, skin-conditioning agents, skin-healing agents, pH adjuster agents, emulsifier agents, surfactant agents, fragrance agents, skin-soothing agents, moisturizing agents, keratolytic agents, antimicrobial agents, antifungal agents, antiviral agents, skin-cleansing agents, skin-regenerative agents, wound healing agents, anti-redness agents, anti-swelling agents, skin-exfoliating agents, and any combination thereof.

12. The method of treatment and/or prevention of claim 9, wherein the administering step involves applying the therapeutically effective amount in form of a liquid gel of the composition or formulation comprising said tattoo aftercare sealant gel as a thin layer over the skin where the tattoo was created, covering all such skin in one go and forming a protective layer over such skin.

13. The method of treatment and/or prevention of claim 9, wherein the protective layer can be easily removed, peeled away, or washed off with water after a period of time following the applying of the composition or formulation comprising said tattoo aftercare sealant gel, and wherein the period of time can range from hours to days post the administering step.

14. The method of treatment and/or prevention of claim 9, wherein the gel-forming-and-bonding components are selected from a group of gel-forming-and-bonding components consisting of: water-soluble or hydrophilic polymers, stabilizers, thickeners, pressure-sensitive adhesives, superabsorbent polymers, softeners, moisturizers, conditioners, emollients, humectants, texture enhancers, solvents, penetration boosters, and any combination thereof.
